(19) United States
(12) Patent Application Publication
(10) Pub. No.: US 2018/0310663 A1
(43) Pub. Date: Nov. 1, 2018

(54) FOOTBED FOR USE IN OPEN FOOTWEAR FOR RELEASABLY RETAINING ORTHOTIC INSERTS

(71) Applicant: Norman BRADLEY, Surrey (CA)

(72) Inventor: Norman BRADLEY, Surrey (CA)

(21) Appl. No.: 15/769,991

(22) PCT Filed: May 10, 2017
(86) PCT No.: PCT/CA2017/000124

§ 371 Date: Apr. 20, 2018

Related U.S. Application Data
(60) Provisional application No. 62/391,881, filed on May 16, 2016.
(60) Provisional application No. 62/357,978, filed on Jul. 2, 2016.

(51) Int. Cl. A43B 7/14; A43B 3/00; A43B 3/12; A43B 17/02; A61F 5/14

(52) U.S. Cl. CPC A43B 7/14; A43B 3/0073; A61F 5/14; A43B 3/0031; A43B 17/02; A43B 3/128

(57) ABSTRACT

A modified footbed is provided for use in an open shoe for releasably retaining an orthotic. The modified footbed comprises: a sole, the sole including a toe end, a heel end, a heel region proximate the heel end, a sole margin and a heel sole margin; and a cover, the cover including a cover margin, a heel cover margin and a heel flap, the sole and the cover attached to one another along the cover margin and the sole margin to define a pocket for accepting an orthotic, the heel flap and the sole at the heel region including at least one mating pair on the heel sole margin and the heel cover margin. An open shoe with the modified footbed is also provided, as is a combination of the open shoe with the modified footbed and an orthotic.

### CROSS REFERENCE TO RELATED APPLICATIONS

This application claims benefit of U.S. Application Ser. No. 62/391,881, filed on May 16, 2016 and entitled SANDAL ORTHOTIC INSERT and U.S. Application Ser. No. 62/357,978, filed on Jul. 2, 2016, entitled FOOTBED FOR USE IN OPEN FOOTWEAR FOR RELEASABLY RETAINING ORTHOTIC INSERTS, the contents of which are incorporated herein by reference.

### FIELD

The present technology relates generally to a modification to footwear to allow for releasable retention of orthotics. The technology more particularly relates to a footbed that includes a pocket for orthotic inserts for use in open footwear such as sandals, flip flops and thong foot wear.

### BACKGROUND

Many people use custom foot supports called orthotics in their shoes to decrease foot pain, improve foot function and balance the biomechanical inadequacies of their feet and legs. Of the 120 million Americans that use orthotics, an estimated 40 million also wear sandals, flip flop, or thong footwear. The problem with orthotic users wearing sandal (open) type footwear without personal orthotics is that it causes discomfort and accelerates foot damage. This causes users to go back to wearing footwear that can accommodate personal orthotics.

Orthotics that can be used in thong type foot wear have been disclosed. For example, US Publication No. 20130318819 discloses a fabric-covered fluid-filled therapeutic foot orthotic with thong-cut device system comprising: a top substrate having fabric covering: a bottom substrate having fabric covering; a high viscosity fluid-filled arch supporting, massaging and comforting-medium; a series of dot welds placed distally of the metatarsal arch; a series of dot welds on a longitudinal arc through the medial arch; a longitudinal swatch weld towards the lateral mid-foot for directional flow and restriction of fluid towards the lateral mid foot; a rearwardly-placed half-moon shaped indentation; a front bladder seal seam; a heel dot weld; and a single forwardly-orientated indented trim line. The design permits the orthotic to be positioned into closed in shoes for transferring further towards the front of the thong cut sandal to provide a ‘correct fit’ without losing any effectiveness of the arch support or therapeutic value while eliminating all unnecessary trim lines. This orthotic simply sits on the foot bed of the footwear and is not retained, nor concealed. As it is not retained, it could simply fall out of place when not weighted down by the user's foot. An unconcealed orthotic is unsightly.

U.S. Pat. No. 7,854,075 discloses an orthotic device that is designed to be reused and reapplied in open shoes include an adhesive layer which securely, but temporarily, bonds the insole to a shoe. In some embodiments, the insole both supports and cushions a foot. In other embodiments, the insole solely cushions the foot. The supporting insoles include an arch support and a heel lift. The cushioning insoles include a flat sole pad, a contoured sole pad, a ball of foot pad and a heel spur pad. In some embodiments, a textile covering is included over the insole. This covering is non-permanently bonded to the rest of the insole. The adhesive layer makes placement of the insole in a closed shoe problematic. Further, the adhesive would have a limited lifespan, and could result in residues being left on the foot beds. The insole is not concealed.

The concept of a pocket for a removable insert in a shoe is disclosed in US Publication No. 20150128451 where the insert is positioned within a pocket formed between the lining of the upper and the exterior shell of the upper. When attached to the upper, the insert forms a part of the exterior surface of the article of footwear. When the insert is removed, part of the pocket forms a portion of the exterior surface of the article of footwear or a flap is extended over the pocket to form a portion of the exterior surface of the article of footwear. The insert may be secured within the pocket using a mechanical fastener. This does not releasably retain an orthotic. It is a sizing system.

What is needed is a footbed for use in an open shoe that releasably retains a personal orthotic. The footbed would preferably have a pocket sized to accept and conceal an orthotic. The pocket would more preferably have at least one closure at the heel end. An open shoe with the modified footbed would also be provided.

### SUMMARY

The present technology is a footbed for use in an open shoe that releasably retains a personal orthotic. The footbed has a pocket sized to accept and conceal an orthotic. The pocket has at least one closure at the heel end. An open shoe with the modified footbed is also provided.

In one embodiment, a modified footbed is provided for use in an open shoe for releasably retaining an orthotic. The modified footbed comprises: a sole, the sole including a heel region proximate a heel end, a heel sole margin at the heel region and a sole margin about a remainder of the sole; and a cover, the cover including a heel cover margin and a heel flap adjacent the heel region and a cover margin about a remainder of the cover, the sole and the cover attached to one another along the cover margin and the sole margin to define a pocket for accepting an orthotic, the heel flap and the sole at the heel region including at least one mating pair on the heel sole margin and the heel cover margin.

In the modified footbed, the mating pair may be a strip of hook and eye closures extending along the heel cover and the heel sole margins.

The modified footbed may further comprise a removable heel insert in the heel region.

In another embodiment, an open shoe for releasably retaining an orthotic is provided, the open shoe including a modified footbed, the modified footbed comprising: a sole, the sole including a heel region proximate a heel end, a heel sole margin at the heel region and a sole margin about a remainder of the sole; and a cover, the cover including a heel cover margin and a heel flap adjacent the heel region and a cover margin about a remainder of the cover, the sole and the cover attached to one another along the cover margin and the sole margin to define a pocket for accepting an orthotic, the heel flap and the sole at the heel region including at least one mating pair on the heel sole margin and the heel cover margin.

The open shoe may further comprise a removable heel insert in the heel region.

In the open shoe, the mating pair may be a strip of hook and eye closures extending along the heel cover and the heel sole margins.

In yet another embodiment, a combination for use by a user in need thereof is provided, the combination comprising an orthotic and an open shoe, the open shoe including a modified footbed, the modified footbed comprising: a sole, the sole including a heel region proximate a heel end, a heel sole margin at the heel region and a sole margin about a remainder of the sole; and a cover, the cover including a heel cover margin and a heel flap adjacent the heel region and a cover margin about a remainder of the cover, the sole and the cover attached to one another along the cover margin and the sole margin to define a pocket for accepting an orthotic, the heel flap and the sole at the heel region including at least one mating pair of releasable closures on the heel sole margin and the heel cover margin.

The open shoe may further comprise a removable heel insert in the heel region.

In the open shoe, the mating pair may be a strip of hook and eye closures extending along the heel cover and the heel sole margins.

### FIGURES

Preferred embodiments of present invention are shown in accompanying figures.

FIG. 1 is a medial longitudinal sectional view of a prior art thong sandal.

FIG. 2 is an exploded perspective view of a modified footbed with the pocket of the present technology.

FIG. 3 is a top view of the sole portion of the modified footbed of the present technology.

FIG. 4 is a bottom view of the cover portion of the modified footbed of the present technology.

### DESCRIPTION

Except as otherwise expressly provided, the following rules of interpretation apply to this specification (written description, claims and drawings): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, The terms “comprising,” “having,” “including,” and “containing” are to be construed as open ended terms (i.e., meaning “including, but not limited to,”) unless otherwise noted.

To the extent necessary to provide descriptive support, the subject matter and/or text of the appended claims is incorporated herein by reference in their entirety.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

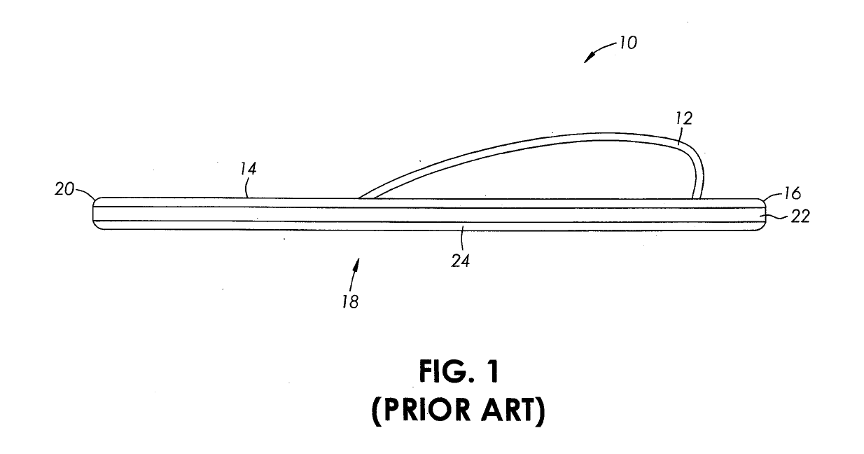

A prior art thong, generally referred to as 10, is shown in FIG. 1. The thong 10 is an exemplary open shoe. As would be known to one skilled in the art, other open shoes, such as, but not limited to, sandals and flip flops are contemplated for use with the present technology—the foot wear may simply have an open heel, or an open toe or openings along the length or any combination thereof. The significance of the shoe type is only that the present invention is designed to allow orthotics to be releasably retained in the modified footbed. Advantageously, the modified fotobed also improves the aesthetics of the combination of an open shoe and an orthotic. The thong 10 has a Y-shaped strap 12 extending from the footbed 14, proximate the toe end 16, to the mid-region, generally referred to as 18. The heel end 20 is open. One or more supporting or padding layers 22 may lie under, and be attached to the footbed 14. An outsole 24 lies under and is attached to either the support or padding layers 22, or if they are absent, the footbed 14. The footbed 14 may be flat or may have contouring.

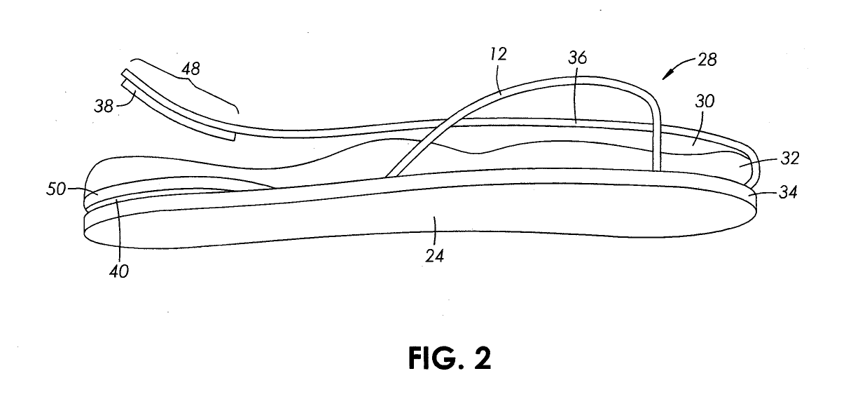

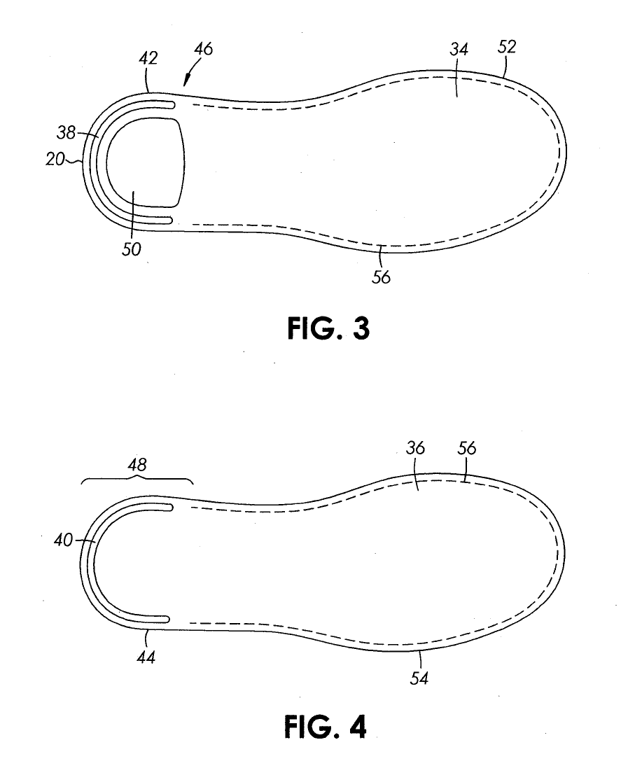

As shown in FIG. 2, a modified footbed, generally referred to as 28 has a pocket 30 that is shaped to conform both to the shape of the open shoe 10 and to an orthotic 32. The pocket 30 is made of a sole 34 below and a cover 36 above. As shown in FIG. 3 and FIG. 4, hook and eye closure mating pairs 38, 40, which is preferably a Velcro® strip, are located on the sole 34 and the cover 36 along the heel sole margin 42 and the heel cover margin 44 proximate the heel end 20 (in the heel region, generally referred to as 46) to form a releasably closeable heel flap 48 (See FIG. 2). Remaining at FIG. 2, the heel flap 48 is opened to insert the orthotic 32, then closed and held closed with the mating pairs 38, 40. When the user wants to remove the orthotic for example, for placement in another shoe, the heel flap 48 is opened and the orthotic 32 is removed. A removable heel insert 50 may be releasably located in the heel region 46, to allow for orthotics with heel attachments. As shown in FIGS. 3 and 4, the cover 36 and the sole 34 are permanently attached to one another around the sole margin 52 and the cover margin 54 by stitching 56, bonding or other method as would be known to one skilled in the art. The sole margin 52 and the cover margin 54 terminate at the heel flap 48. The cover 36 is leather, synthetic, fabric or any other material that would be contemplated for use in a shoe.

The footbed 28 can be used in the manufacturing of any open shoe. One or more supporting or padding layers 22 may lie under, and be attached to the modified footbed 28. An outsole 24 lies under and is attached to either the support or padding layers 22, or if they are absent, the modified footbed 28. The method of using the footbed 28 is outlined above.

While the particular device has been illustrated and described in detail with the help of drawings and foregoing descriptions, such an illustration and description is to be considered as exemplary and not restrictive in character, and is to be understood that it is the presently preferred embodiments of the present technology and is thus representative of the subject matter which is broadly contemplated by the present technology, and that all changes and modifications that come within the spirit of the disclosure are desired to be protected.

1. A modified footbed for use in an open shoe for releas ably retaining an orthotic, the modified footbed comprising: a sole, the sole including a heel region proximate a heel end, a heel sole margin at the heel region and a sole margin about a remainder of the sole; and a cover, the cover including a heel cover margin, a heel flap adjacent the heel region and a cover margin about a remainder of the cover, the sole and the cover attached to one another along the cover margin and the sole margin to define a pocket for accepting an orthotic, the pocket shaped to conform to the orthotic, the heel flap and the sole at the heel region including at least one mating pair of releasable closures on the heel sole margin and the heel cover margin.

2. The modified footbed of claim 1, wherein the at least one mating pair is a strip of hook and eye closures extending along the heel cover and the heel sole margins.

3. The modified footbed of claim 1, further comprising a removable heel insert in the heel region.

4. An open shoe for releasably retaining an orthotic, the open shoe including a modified footbed, the modified footbed comprising: a sole, the sole including a heel region proximate a heel end, a heel sole margin at the heel region and a sole margin about a remainder of the sole; and a cover, the cover including a heel cover margin, and a heel flap adjacent the heel region and a cover margin about a remainder of the cover, the sole and the cover attached to one another along the cover margin and the sole margin to define a pocket for accepting an orthotic, the pocket shaped to conform to the orthotic, the heel flap and the sole at the heel region including at least one mating pair of releasable closures on the heel sole margin and the heel cover margin.

5. The open shoe of claim 4, further comprising a removable heel insert in the heel region.

6. The open shoe of claim 5, wherein the at least one mating pair is a strip of hook and eye closures extending along the heel cover and the heel sole margins.

7. A combination for use by a user in need thereof, the combination comprising an orthotic and an open shoe, the open shoe including a modified footbed, the modified footbed comprising: a sole, the sole including a heel region proximate a heel end, a heel sole margin at the heel region and a sole margin about a remainder of the sole; and a cover, the cover including, a heel cover margin a heel flap adjacent the heel region and a cover margin about a remainder of the cover, the sole and the cover attached to one another along the cover margin and the sole margin to define a pocket for accepting an orthotic, the pocket shaped to conform to the orthotic, the heel flap and the sole at the heel region including at least one mating pair of releasable closures on the heel sole margin and the heel cover margin.

8. The open shoe of claim 7, further comprising a removable heel insert in the heel region.

9. The open shoe of claim 7, wherein the at least one mating pair are a strip of hook and eye closures extending along the heel cover and the heel sole margins.

10. The modified footbed of claim 3, wherein the cover is leather.

11. The modified footbed of claim 10, wherein the pocket is additionally shaped to conform to the open shoe.
